(19) United States
(12) Patent Application Publication
(10) Pub. No.: US 2005/0042129 A1
(43) Pub. Date: Feb. 24, 2005

(54) Method and apparatus for irradiating fluids

(76) Inventor: Bijan Kazem, Woodstock, GA (US)

Correspondence Address: WOMBLE CARLYLE SANDRIDGE & RICE, PLLC, P.O. BOX 7037, ATLANTA, GA, 30357-0037, US

(21) Appl. No.: 10/919,064

(22) Filed: Aug. 16, 2004

Related U.S. Application Data
(60) Provisional application No. 60/497,057, filed on Aug. 22, 2003.

(51) Int. Cl. A61L 2/025; A61L 2/10

(52) U.S. Cl. 422/20; 422/127; 250/435; 422/23; 422/24

(57) ABSTRACT

A method and an apparatus for treating fluids are provided. The method generally includes cavitating and irradiating a liquid. The irradiation of the liquid may include exposing the liquid to ultraviolet radiation. The apparatus generally includes a housing having a chamber formed therein and defined, at least in part, by a chamber wall that transmits radiation therethrough. The apparatus also includes a cavitator in flow communication with the interior of the chamber and a radiator aligned to direct radiation into the interior of the chamber. Cavitation generated by the apparatus and/or provided in the method tends to refresh the liquid exposed to the radiationt, thereby increasing the rate of radiation exposure for the liquid.

### CROSS REFERENCE TO RELATED APPLICATIONS

The present application claims priority to the filing date of U.S. Provisional Patent Application Ser. No. 60/497,057, filed Aug. 22, 2003, which is incorporated by reference as if set forth herein in its entirety.

### TECHNICAL FIELD

The disclosure generally relates to irradiating fluids and more specifically to methods and devices for cavitating a fluid while exposed to radiation.

### BACKGROUND

Liquids frequently contain contaminants, such as microorganisms and toxic compounds, which may prove harmful in subsequent uses. Examples of microorganisms frequently found in liquids include bacteria, spores, yeasts, fungi, algae, and viruses or bacteriophages. Toxic compounds found in liquids may include cancer-causing aromatic compounds and numerous halogen compounds, particularly chlorine compounds.

There are many known techniques for disinfecting liquids, including the use of chemical or physical agents, mechanical means, and radiation. The traditional method of disinfection has been the use of chemical agents in the form of chlorine. Although chlorine disinfection has significantly reduced the incidence of waterborne disease, there is growing concern about chlorine's safety. Mechanical means often include expensive machinery that involves substantial capital costs and upkeep.

Radiation disinfection includes the breaking of chemical bonds under the action of the ultraviolet (UV) radiation through photodissociation. A particular substance will have a characteristic photodissociation curve associated with it specifying the energies and wavelengths of UV radiation for which the particular substance will undergo photodissociation. For effective photodissociation, it is necessary that the UV radiation have the particular energy or energies which fall within the photodissociation curve of the substance of interest.

With respect to microorganisms, disinfection occurs when UV light contacts the microorganism's deoxyribonucleic acid (DNA) molecules, which contain the genetic information necessary for cell replication. The light causes double bonds to form between adjacent subgroups in the DNA structure, preventing normal replication of DNA molecules and thereby inactivating the microorganism.

Most existing radiation disinfecting systems pump liquid through pipes lined with dozens of UV lamps. However, the lamps tend to foul quickly, reducing their effectiveness and requiring ongoing cleaning and replacement. Additionally, UV radiation has little penetrating power such that the liquid stream must be run through long pipes to increase the likelihood that UV radiation will contact enough of the liquid to affect the microorganisms it carries.

### SUMMARY

Briefly described, methods and apparatus for treating fluids are provided. The methods and apparatus generally provide for the treatment of fluids, particularly liquids, with cavitation and irradiation. The combination of cavitation and irradiation can allow for more complete irradiation of the fluid in a shorter period of time and higher efficiencies than would be available with some other methods and apparatus.

In one aspect of the present invention, a method of treating a liquid is provided which comprises introducing a liquid into a chamber, creating cavitation in the chamber and irradiating the liquid in the presence of cavitation in the chamber.

In another aspect of the present invention, a method of treating a liquid is provided in which a liquid is mechanically cavitated and irradiated with ultraviolet radiation.

In still a further aspect of the present invention, an apparatus for treating a fluid is provided which comprises a housing having a chamber formed therein. The chamber comprises at least one chamber wall defining at least a portion of an interior of the chamber. The apparatus also comprises a cavitator disposed in the chamber, and a radiator separated from the interior of the chamber by the chamber wall and aligned to direct radiation into the chamber. The chamber wall is capable of transmitting radiation generated by the radiator to the interior of the chamber, thereby allowing a fluid, such as a liquid, contained in the chamber to be irradiated.

In still another aspect of the present invention, an apparatus for treating a fluid is provided which comprises a housing having an outer wall that is translucent and a cavitator disposed in the housing. A radiator is aligned so as to direct radiation into the housing through the outer wall so as to irradiate the contents of the housing.

In still a further aspect of the present invention, an apparatus for treating fluids is provided that comprises a housing having a chamber formed therein and a mechanical cavitator in flow communication with the chamber. A radiator also is provided an aligned to direct radiation to the chamber.

These and other aspects of are set forth in greater detail below and shown in the drawings which are briefly described as follows.

### BRIEF DESCRIPTION OF THE DRAWINGS

FIG. 1 illustrates an apparatus for treating a fluid.

FIG. 2 is a cross-sectional view of the reactor illustrated in FIG. 1.

FIG. 3 is a graph of the absorbance of liquid irradiated in an apparatus encompassing aspects of the present invention.

FIG. 4 is another graph of the absorbance of liquid irradiated in an apparatus encompassing aspects of the present invention.

### DETAILED DESCRIPTION

Methods and apparatus for irradiating and cavitating fluids are disclosed. As used herein, the term “irradiating” refers to both emitting and/or casting upon something radiation that includes monochromatic light, visible light, gamma rays, X-rays, ultraviolet, infrared, microwaves, and radio waves. The term “cavitating” refers to the formation of at least partial vacuums in a fluid, such as a liquid. The term “mechanically cavitating” is limited to the formation of at least partial vacuums in a fluid, such as a liquid, by swiftly moving one or more bodies through the fluid. In the methods and apparatus, a liquid is introduced into a cavitation zone, which can be within a chamber, where it is cavitated and also irradiated.

The fluids, such as liquid, gas/liquid, and gas streams, to be treated can be virtually any stream that can flow through the system. In the food and drink industry, where there is a concern about the existence of pathogens, the liquid stream may comprise water, soft drinks, brewing, dairy products and fruit juices. Additionally, the liquid stream may comprise petrochemicals such as in the photopolymerization of vinyl monomers, N,N-dimethylacrylamide, Poly N-Isopropylacrylamide (NIPAAM/X) and any thermally sensitive polymerization. Furthermore, the liquid stream may be a liquid formed in the paper and pulp industry, wherein the radiation reduces the viscosity of the liquid.

The radiation source 10 may be any device capable of producing an electromagnetic radiation. Various spectrums of electromagnetic radiation may be utilized. Example spectrums include ultraviolet, microwave, gamma ray, monochromatic light and the visible light spectrum. The absorption of the electromagnetic radiation by the liquid stream can produce a chemical change in the liquid stream. Without being bound to any particular theory, it is surmised that such photochemical reactions or changes proceed via interactions between photons and single molecules. Example reactions include the dissociation of O—H, C—H and C—C bonds which aid in the disinfection liquid streams that may be contaminated with pathogens.

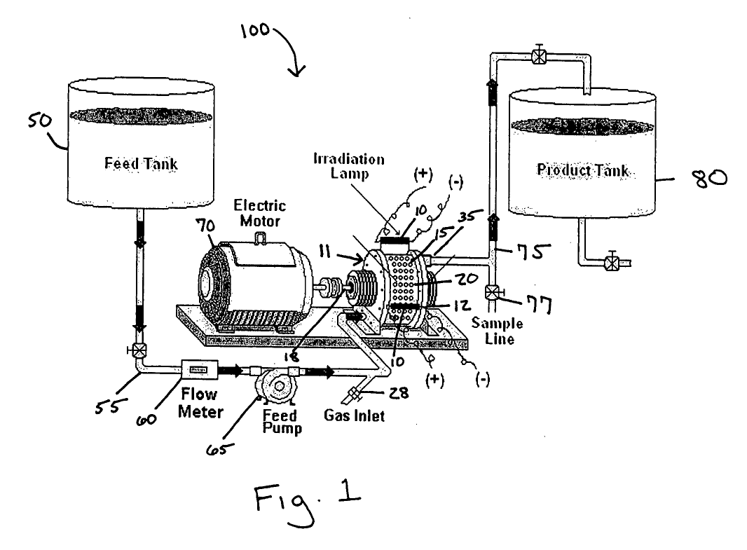

Referring now in more detail to the drawings, in which like numerals refer to like parts throughout the several views, FIG. 1 illustrates a system 100 comprising an apparatus in which a fluid can be irradiated and cavitated. The apparatus is referred to herein as a reactor 11. The system 100 also includes a feed tank 50 which contains the liquid that is to be treated. The feed tank 50 is in flow communication with the reactor 11 by delivery line 55, which has a flow meter 60 disposed therein for monitoring the flow rate and/or amount of liquid flowing through the delivery line 55. A feed pump 65 also is provided in flow communication with the delivery line 55 to pump the liquid from the feed tank 50 to the reactor 11. A gas inlet 28 is provided in flow communication with the delivery line 55 to allow the introduction of gaseous components into the liquid stream as it flows to the reactor 11.

An electric motor 70 is operably connected to the shaft 18 of the cavitator 20 so as to provide the driving force for rotating the rotor 17 of the cavitator 20. As used herein, the term “cavitator” refers to a device that can induce cavitation in a fluid. Also, as used herein, the term “mechanical cavitator” refers to a device that induce cavitation in a fluid by moving a body through the fluid. A product line 75 is in flow communication with the reactor 11 and routes the treated fluids to a product tank 80. A sample line 77 can be provided inline with the product line 75 to allow samples for quality testing to be easily removed from the system 100.

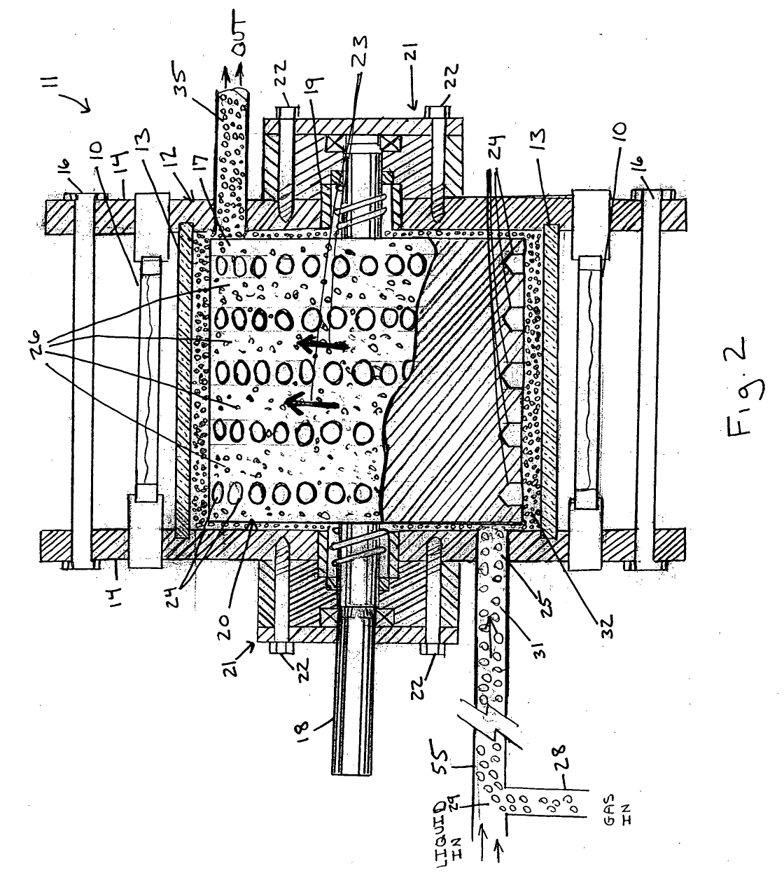

As shown in FIGS. 1 and 2, the reactor 11 comprises a cylindrical housing 12 defining an internal cylindrical chamber 15. In the figures, the housing 12 is formed of a wall 13 capped by end plates 14 secured to each other by bolts 16. The wall 13 is sandwiched between the plates 14.

The radiator 10 is positioned such that the liquid stream in the cavitation zone is in contact with the irradiation emitted from the radiator. As shown in FIGS. 1 and 2, the radiator 10 is a ultraviolet lamp mounted to the housing 12 of the reactor 11. The reactor 11 can include a plurality of radiators 10 mounted to or aligned therewith so as to direct irradiation at the fluids in the housing 12, particularly the fluids in the cavitation zone 32. The radiator 10 may include any device capable of producing electromagnetic radiation.

Typically, the radiator 10 is separated from the interior of the chamber 15 by the chamber wall 13, which is substantially transparent to the irradiation generated by the radiator 10, such that the chamber wall transmits the radiation generated by the radiation source 10 into the chamber 15 so that the liquid in the chamber is irradiated. The radiator 10 may be placed within the housing 12, but generally is separated from the interior of the chamber 15 by the chamber wall 13, so that the radiator does not come in direct contact with the liquid being treated. This separation of the radiator and the liquid reduces the possibility of contamination of the liquid by a malfunctioning or broken radiator and reduces the frequency of fouling of the radiator by the liquid, thereby potentially reducing the costs of maintaining the system.

The wall 13 that transmits the radiation generated by the radiator 10 can be formed of a translucent material, such as silica compounds, like quartz and fused silica, polycarbonates, polytetrafluoroethylenes and other translucent materials. The wall 13 may be cylindrical, as shown in FIGS. 1 and 2, or be formed of plates of translucent or otherwise radiation transmitting material that make up all or a portion of the outer wall of the housing 12. It is contemplated that the outer wall of the housing can include both translucent and non-translucent sections, wherein the radiators are aligned with the translucent sections to direct radiation into the interior of the chamber of the housing.

The dosage and intensity of the irradiation is varied depending upon the contents of the liquid stream and the desired level of treatment of the stream. Although the radiators 10 are shown in FIGS. 1 and 2 disposed outside the interior of the housing 12, it is contemplated that one or more radiators 10 can be disposed within the housing 12 but separated from the interior of the chamber 15 by an interior chamber wall that is translucent or otherwise transmits radiation.

The cylindrical rotor 17 is disposed within the cylindrical chamber 15 of the housing and is mounted on the axially extending shaft 18. The shaft 18 is journaled on either side of the rotor within bearing assemblies 19 that, in turn, are mounted within bearing assembly housings 21. The bearing assembly housings 21 are secured to the housing 12 by means of appropriate fasteners such as bolts 22. The shaft 18 projects from one of the bearing housings 21 and is coupled to the electric motor 70 or other motive means. It will thus be seen that the rotor 17 may be spun or rotated within the cylindrical chamber 15 in the direction of arrows 23 by activating the motor 70 coupled to the shaft 18.

The rotor 17 has a peripheral surface that is formed with one or more circumferentially extending arrays of irregularities in the form of relatively shallow holes or bores 24. As shown in FIG. 2, the rotor 17 is provided with five arrays of bores 24 separated by voids 26, the purpose of which is described in more detail below. It should be understood, however, that fewer or more than five arrays of bores may be provided in the peripheral surface of the rotor as desired depending upon the intended fluids and flow rates. Further, irregularities other than holes or bores also may be provided. The rotor 17 is sized relative to the cylindrical chamber 15 in which it is housed to define a space, referred to herein as a cavitation zone 32, between the peripheral surface of the rotor and the cylindrical chamber wall 13 of the chamber 15.

An inlet port 25 is provided in the endplate 14 of housing 12 for supplying from the delivery line 55 fluids to be treated to the interior chamber 15 within the housing. Gas supply from the gas supply conduit 28 is introduced and entrained in the form of bubbles within the stream of liquid flowing through the delivery line 55, if desired.

In the case of a liquid to be oxidized in the presence of an oxidizer, such as ozone, liquid is pumped through the delivery line 55 from the feed tank 50 and ozone, which contains oxygen and ozone, is supplied through the gas supply conduit 28. At the junction of the delivery line 55 and the gas supply conduit 28, the liquid and ozone form a gas/liquid mixture in the form of relatively large ozone bubbles 31 entrained within the flow of liquid 29. This mixture of liquid and ozone bubbles is directed into the cylindrical chamber 15 of the housing 12 through the inlet port 25 as shown.

An outlet port 35 is provided in the endplate 14 of housing 12 and is located opposite to the location of the inlet port 25. Location of the outlet port 35 in this way ensures that the entire volume of the gas/liquid mixture traverses at least one of the arrays of bores 24 and thus moves through a cavitation zone prior to exiting the reactor 11. The outlet port 35 is formed in the endplate 14 of the housing 12 and is in fluid communication with the product line 75 so as to allow treated fluids to be delivered to a collection area, such as product tank 80.

In operation, the reactor 11 functions to cavitate and irradiate a fluid, which can be used to oxidize environmentally harmful compounds within a liquid. A liquid containing environmentally harmful compound is pumped through the delivery line 55. In order to enhance the effect of the radiation on the liquid stream, a flow of oxidant can be interjected into the liquid. A gaseous oxidant, such as ozone, is supplied through the gas supply conduit 28 to the stream of liquid and the air and liquid form a mixture comprised of relatively large ozone bubbles 31 entrained within the liquid 29. The liquid/ozone bubble mixture moves through the delivery line 55 and enters the chamber 15 through the supply port 25.

From the supply port 25, the mixture moves toward the periphery of the rapidly rotating rotor 17 and enters the cavitation zones 32 in the region of the bores 24. As described in substantial detail in our previously issued U.S. Pat. No. 6,627,784, the disclosure of which is hereby incorporated by reference, within the cavitation zones 32, millions of microscopic cavitation bubbles are formed in the mixture within and around the rapidly moving bores 24 on the rotor. Since these cavitation bubbles are unstable, they collapse rapidly after their formation. As a result, the millions of microscopic cavitation bubbles continuously form and collapse within and around the bores 24 of the rotor, creating cavitation induced shock waves that propagate through the mixture in a violent albeit localized process.

As the mixture of liquid and relatively large ozone bubbles moves into and through the cavitation zones 32, the ozone bubbles in the mixture are bombarded by the microscopic cavitation bubbles as they form and further are impacted by the cavitation shock waves created as the cavitation bubbles collapse. This results in a “chopping up” of the relatively large ozone bubbles into smaller bubbles, which themselves are chopped up into even smaller air bubbles and so on in a process that occurs very quickly. Thus, the original ozone bubbles are continuously chopped up and reduced to millions of tiny microscopic ozone bubbles within the cavitation zone.

The dispersement and random flow patterns within the cavitation zone 32 provide a high degree of mixing of the oxidant and liquid/gas streams. Some conventional systems do not achieve a thorough mixing of the oxidant and liquid/gas streams, thus requiring the addition of substantially more oxidant and/or radiation into the liquid stream, resulting in increased costs and still not guaranteeing even mixing of the combination. The turbulence of the fluids within the cavitation zone 32 leads to more complete mixing of the oxidant with the liquid.

The agitation of the liquid resulting from the cavitation causes the liquid at the surface of the wall 13 to be refreshed at a very high rate. A high rate of liquid surface refreshing at the wall 13 increases the exposure of the liquid to the radiation transmitted through the wall 13 from the radiators 10. This refreshing aids in introducing a greater surface area of fluid to the radiation treatment zone. Even with opaque liquids, such as dairy milk and black liquor, the cavitation induced in the liquid increases the rate of exposure of the liquid to the radiation. Upon interaction with radiation, such as the UV radiation generated by the radiators 10, and/or the gas stream, free radicals are created which chemically react with contaminants in the gas and/or liquid streams.

The term “cavitation zone” is used herein to refer to the region between the outer periphery of the rotor wherein the bores are formed and the cylindrical wall of the housing chamber. This is where the most intense cavitation activity occurs. It should be understood, however, that cavitation may occur, albeit with less intensity, in regions other than this space such as, for example, in the reservoir or region between the sides or faces of the rotor and the housing.

The process of cavitating and irradiating a fluid can be on a substantially continuous basis in that a continuous flow of liquid is pumped into the reactor 11, treated by cavitation and irradiation and then discharged from the reactor 11. Alternatively, the reactor 11 can be configured to treat fluids on a batch wise basis, wherein a specified amount of liquid is charged to the reactor 11, treated by cavitation and irradiation, and then discharged before any additional material is charged to the reactor.

In another example, liquid, such as water, contaminated with dioxins, cyclic toxics or halogenated contaminants, such as chlorinated organic molecules (e.g., trichloroethylene, vinylidene chloride and vinyl chloride) can be treated by cavitating and irradiating the liquid with UV radiation in the presence of an oxidant, such as hydrogen peroxide (H₂O₂). When hydrogen peroxide comes into contact with UV light, hydroxyl radicals are produced that attack the UV unsaturated bonds in dioxins and cyclic toxics forming less hazardous compounds.

A supply of water is channeled to the reactor 11 and mixed with hydrogen peroxide (or other suitable oxidant). The liquid combination is then channeled into the reactor 11 where it is cavitated in the chamber 15 of housing 12. The radiators 10, in the form of UV lamps are arranged around the perimeter of the chamber 15 and separated from the interior of the chamber by wall 13. The radiation generated by the radiators 10 are transmitted through the wall 13 and irradiate the water and hydrogen peroxide mixture thereby producing hydroxyl radicals. The hydroxyl radicals that are formed attack the halogenated compounds in the stream and chemically convert it to a more favorable substance, such as carbon dioxide and water. The UV light also operates to kill bacteria within the water.

In another aspect of the present invention, an apparatus for treating fluids is provided that includes one or more radiators that direct radiation to a chamber formed in a housing of the apparatus. The apparatus includes a cavitator arranged to provide cavitation to the fluid in the chamber of the apparatus. The radiator can be disposed inside the housing, and even inside the chamber itself. In these instances, one or more walls of the chamber can be reflective to facilitate the focusing of the radiation into the cavitation zone in the chamber. The reflective wall(s) of the chamber can include aluminum, mirrors or other reflective materials.

### EXAMPLES

### Example 1

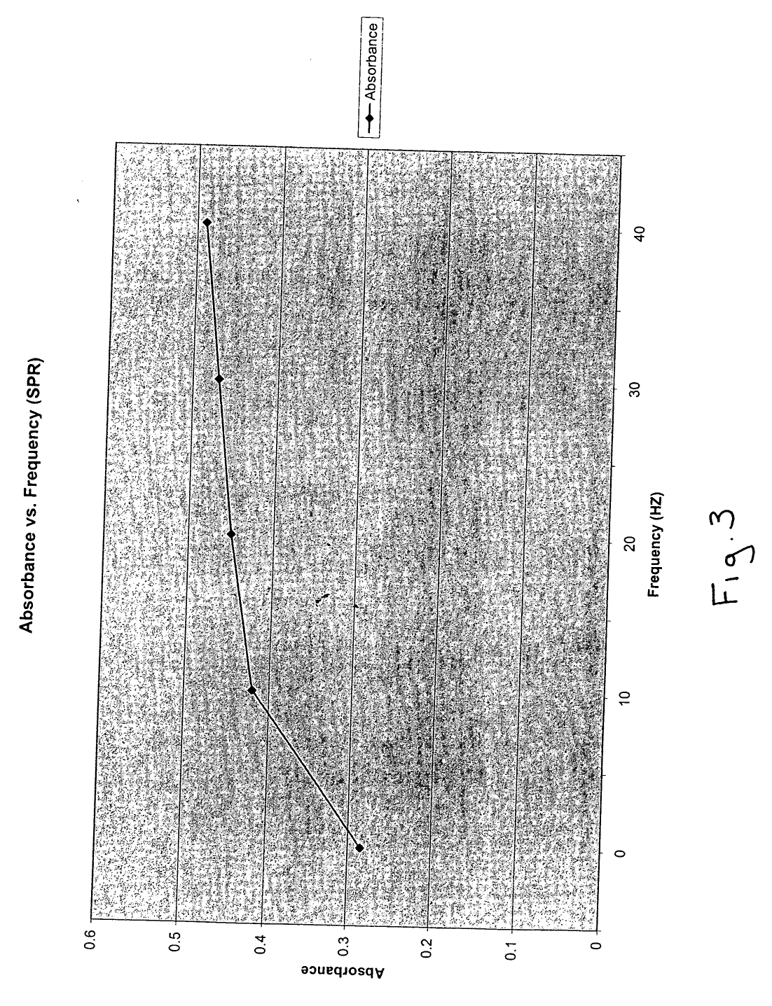

An aqueous solution containing 0.03 M KI and 0.005 M KIO₃ was fed into a reactor that included a cavitator. Potassium iodide was included in the solution because it's color changes as it is oxidized, thereby showing the extent of reaction in each sample run. The pH of the solution was approximately 9.25. The absorbance of the liquid then was determined and the percent transmission calculated at 350 nanometers (nm). The reactor included a rotor with dimensions of 6 inches by 1.5 inches and a housing with a 7.75 inch outer diameter with the translucent wall made of 19 mm thick quartz. The rotor-to-housing clearance was 0.125 inches and the rotor-to-endplate clearance was 0.75 inches. The reactor included four UV lamps aligned around the translucent quartz chamber wall of the housing. Each UV lamp had a wattage of 18 watts nominal and a photon wattage expressed as intensity at one meter of 42 microwatts/cm². The pH of the solution was approximately 9.18. Multiple runs were conducted at about 1.5 l/min. in which the frequency of the rotor was increased by 10 Hz for each successive sample. The results are shown in Table 1 and a graphical representation of the absorbance of the liquid versus the frequency of the rotor is shown in FIG. 3.

TABLE 1

| Tin | Tout | Pressure | UV | Frequency | Sample | Absorbance | % Transmission |
|---|---|---|---|---|---|---|---|
| ° F. | ° F. | Psig | Lamps | HZ | # | at 350 nm | at 350 nm |
| 80.7 | 80.3 | 20 | 4 | 0 | 1 | 0.285 | 51.7 |
| 80.9 | 81.1 | 20 | 4 | 10 | 2 | 0.42 | 38 |
| 80.8 | 82.5 | 20 | 4 | 20 | 3 | 0.45 | 35.5 |
| 80.9 | 84.1 | 20 | 4 | 30 | 4 | 0.47 | 34.1 |
| 80.9 | 87.3 | 20 | 4 | 40 | 5 | 0.49 | 32.8 |

As shown in Table 1 and FIG. 3, the absorbance increased with each increase in the frequency of the rotor. A significant increase in the absorbance is shown to occur between 0 Hz and 10 Hz. This increase reflects the effect of increasing the refresh rate of the surface of the liquid exposed to the radiation has on the completeness of the reaction.

### Example 2

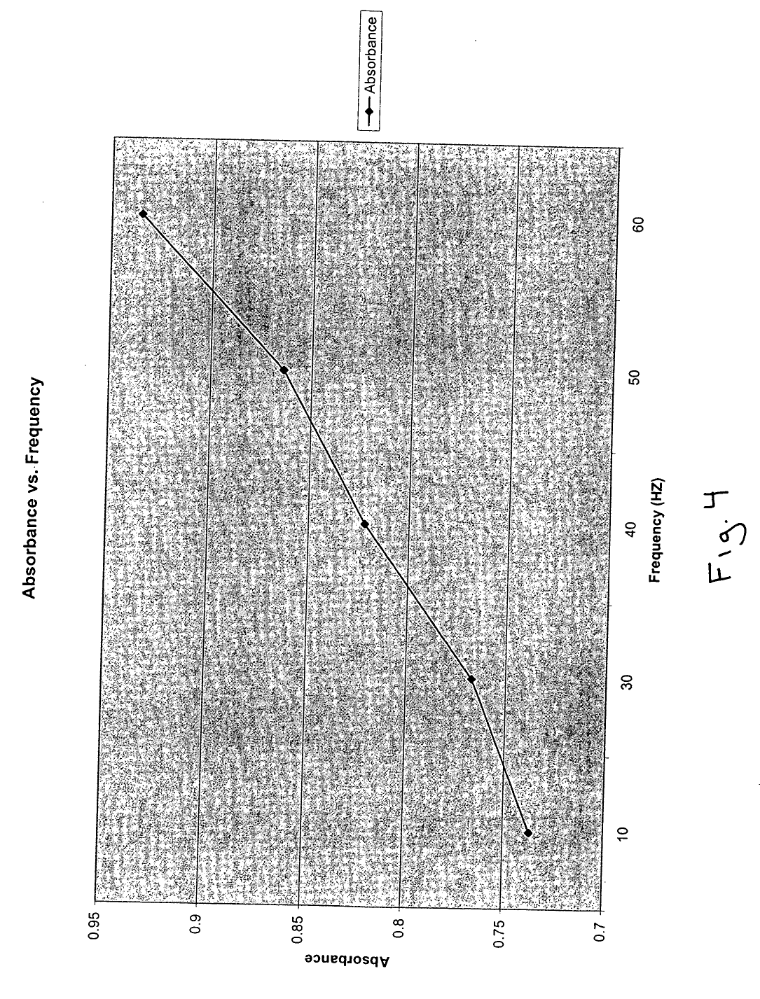

An aqueous solution containing 0.3M potassium iodide (KI) and 0.05 M potassium iodate (KIO₃) was fed into a reactor as described in Example 1. The pH of the solution was approximately 9.25. Six runs of this solution in this reactor were run at about 1500 ml/min. In each successive run or sample, the frequency of the rotor was increased by 10 HZ or 600 revolutions per minute (rpm). The absorbance of the liquid was determined and the percent transmission calculated at 350 nanometers (nm). The results are shown in Table 2 and the correlation between the frequency of the rotor of the reactor and the absorbance of the liquid is shown graphically in FIG. 4.

TABLE 2

| Tin | Tout | Pressure | UV | Frequency | Sample | Absorbance | % Transmission |
|---|---|---|---|---|---|---|---|
| ° F. | ° F. | Psig | Lamps | HZ | # | at 350 nm | at 350 nm |
| 78.2 | 77.2 | 20 | 4 | 10 | 1 | 0.737 | 18.3 |
| 78.1 | 79.8 | 20 | 4 | 30 | 3 | 0.767 | 17.1 |
| 78.7 | 83.8 | 20 | 4 | 40 | 4 | 0.822 | 15.1 |
| 79 | 87.6 | 20 | 4 | 50 | 5 | 0.864 | 13.7 |
| 78.9 | 89.1 | 20 | 4 | 60 | 6 | 0.935 | 11.7 |

As can be seen, the absorbance increased and the percent transmission decreased as the frequency of the rotor increased. Without being limited to a particular theory, it is surmised that an increase in the frequency or rotation of the rotor leads to an increased rate of cavitation induced in the liquid in the reactor. It is surmised that an increased rate of cavitation generated in the liquid in the reactor increases the refresh rate of liquid brought to the chamber wall of the mixer, thereby increasing the rate of liquid exposed to the radiation from the UV lamps, and thereby increasing the extent of oxidation or other reaction, which results in the liquid displaying increased absorbance and lower percent transmission values.

Although certain aspects of the invention have been described and illustrated, it should be understood by those skilled in the art that the foregoing and various other changes, omissions and additions may be made therein and thereto, without parting from the spirit and scope of the present invention.

1. An apparatus for treating a fluid comprising:
a housing having a chamber formed therein, wherein said chamber comprises at least one chamber wall defining at least a portion of an interior of said chamber;
a cavitator disposed in said chamber; and,
a radiator separated from said interior by said chamber wall and aligned to direct radiation into said chamber, wherein said chamber wall transmits radiation generated by said radiator to said interior of said chamber.

2. The apparatus of claim 1, wherein said housing further comprises a liquid port in flow communication with said interior of said chamber.

3. The apparatus of claim 1, wherein said radiator is mounted to said housing.

4. The apparatus of claim 1, wherein said cavitator comprises a rotor.

5. The apparatus of claim 1, wherein said cavitator comprises a body having a plurality of bores formed therein.

6. The apparatus of claim 1, wherein said chamber wall is translucent.

7. The apparatus of claim 1, wherein said radiator is capable of generating ultraviolet radiation.

8. The apparatus of claim 7, wherein said radiator comprises an ultraviolet lamp.

9. The apparatus of claim 1, wherein said chamber wall is an outer wall of said housing.

10. The apparatus of claim 1, wherein said radiator comprises a plurality of radiation sources distributed around said housing.

11. The apparatus of claim 1, further comprising a gas port in flow communication with said interior of said chamber.

12. An apparatus for treating a fluid comprising:
a housing having an outer wall, wherein said outer wall is translucent;
a cavitator disposed in said housing; and
a radiator aligned to direct radiation into said housing through said outer wall.

13. The apparatus of claim 12, wherein said cavitator comprises a rotor having a plurality of bores formed therein.

14. The apparatus of claim 11, wherein said radiator is capable of generating ultraviolet radiation.

15. The apparatus of claim 14, wherein said radiator comprises an ultraviolet lamp.

16. The apparatus of claim 11, wherein said radiator is mounted to said housing.

17. A method of treating a liquid comprising:
introducing a liquid into a chamber;
cavitating the liquid in the chamber; and,
irradiating the liquid in the chamber.

18. The method of claim 17, wherein cavitating the liquid comprises moving a body through the liquid.

19. The method of claim 17, wherein irradiating the liquid comprises directing ultraviolet radiation at the liquid.

20. The method of claim 17, wherein introducing the liquid into the chamber is substantially continuous.

21. The method of claim 17, further comprising introducing a gas into the liquid.

22. The method of claim 17, further comprising oxidizing a component of the liquid in the chamber.

23. The method of claim 17, wherein the liquid is substantially opaque.

24. A method of treating a liquid comprising:
mechanically cavitating a liquid; and
irradiating the liquid with ultraviolet radiation.

25. The method of claim 24, further comprising oxidizing a component of the liquid.

26. The method of claim 24, wherein the liquid is substantially opaque.

27. The method of claim 24, wherein mechanically cavitating the liquid and irradiating the liquid are substantially continuous.

28. An apparatus for treating fluids comprising:
a housing having a chamber formed therein;
a mechanical cavitator in flow communication with said chamber; and
a radiator aligned to direct radiation to said chamber.

29. The apparatus of claim 28, wherein said cavitator comprises a rotor.

30. The apparatus of claim 28, wherein said cavitator comprises a body having a plurality of bores formed therein.

31. The apparatus of claim 28, wherein the radiator is capable of generating ultraviolet radiation.
